(19) United States
(12) Patent Application Publication
(10) Pub. No.: US 2015/0216923 A1
(43) Pub. Date: Aug. 6, 2015

(54) Biochemical Scaffolds for Modulating Cell Function

(71) Applicants: Ralph L. Peterson, Oakland, CA (US); Renee D. Williman, Oakland, CA (US); Seth L. Forster, Vernal, UT (US)

(72) Inventors: Ralph L. Peterson, Oakland, CA (US); Renee D. Williman, Oakland, CA (US); Seth L. Forster, Vernal, UT (US)

(21) Appl. No.: 14/223,392

(22) Filed: Mar. 24, 2014

Related U.S. Application Data
(60) Provisional application No. 61/936,116, filed on Feb. 5, 2014.

(51) Int. Cl. A61K 36/79; A61K 36/31; A61K 31/198; A61K 31/525; A61K 31/455; A61K 31/191; A61K 31/519; A61K 36/296; A61K 36/185; A61K 33/26; A61K 33/34; A61K 31/675; A61K 31/4188; A61K 36/74; A61K 31/714; A61K 31/555; A61K 33/06; A61K 33/32; A61K 41/00; A61H 23/00; A61K 36/254

(52) U.S. Cl. CPC A61K 36/79; A61K 36/254; A61K 36/31; A61K 31/198; A61K 31/525; A61K 31/455; A61K 31/191; A61K 31/519; A61K 36/296; A61K 36/185; A61K 33/26; A61K 33/34; A61K 31/675; A61K 31/4188; A61K 36/74; A61K 31/714; A61K 31/555; A61K 33/06; A61K 33/32; A61K 41/00; A61H 23/00

(57) ABSTRACT

A biochemical scaffold for regulating mammalian cell function, including a bioenergetic platform and a vibrational platform, the bioenergetic platform comprising a Krebs cycle modulator, glutathione modulator, neurotransmitter modulator and a DNA modulator, the vibrational platform comprising a plurality of herbs that are subjected to harmonic oscillation in the range of approximately 102 GHz-250 GHz for a period of time in the range of approximately 3-5 hrs.

### CROSS-REFERENCES TO RELATED APPLICATIONS

This application claims the benefit of U.S. Provisional Application No. 61/936,116, filed on Feb. 5, 2014.

### FIELD OF THE INVENTION

The present invention relates to compositions and methods for regulating cell function. More particularly, the present invention relates to biochemical scaffolds and associated methods for modulating a plurality of cell activities and functions.

### BACKGROUND OF THE INVENTION

As is well known in the art, optimal cell activity and, hence, function is essential to human existence. Cell activity and function is primarily dependent on the energy potential of a cell. Where cellular energy has been reduced, a cascade of undesirable cellular events can, and often times will, result. The noted cellular events typically result in one or more undesirable physiological characteristics, such as reduced stamina or endurance, and mental clarity.

Reduction of cellular energy can also result in dysfunction of various organs, e.g., heart and/or liver failure. When cellular energy approaches zero, cell death, i.e. apoptosis, is often encountered.

As is also well known in the art, cellular energy is directly dependent on various biochemical processes; particular, cell respiration, i.e. metabolic reactions and processes that take place in the cells to convert biochemical energy from nutrients into adenosine triphosphate (ATP).

The metabolic reactions and processes, which are often referred to as a metabolic pathway, are typically embodied in the Krebs cycle.

Referring to FIG. 1, there is shown a schematic illustration of a Krebs cycle. As illustrated in FIG. 1, through catabolism of carbohydrates, fats and proteins, a two carbon organic product, i.e. acetate in the form of acetyl-CoA, is produced. Acetyl-CoA and two equivalents of water (H₂O) are consumed during the citric acid cycle, producing two equivalents of carbon dioxide (CO₂) and one equivalent of HS-CoA.

In addition, one complete evolution of the Kreb cycle converts three equivalents of nicotinamide adenine dinucleotide (NAD⁺) into three equivalents of reduced NAD⁺ (NADH), one equivalent of ubiquinone (Q) into one equivalent of reduced ubiquinone (QH₂), and one equivalent each of guanosine diphosphate (GDP) and inorganic phosphate (Pᵢ) into one equivalent of guanosine triphosphate (GTP). The NADH and QH₂ generated during the Kreb cycle are in turn used by the oxidative phosphorylation pathway to generate energy-rich adenosine triphosphate (ATP).

A primary source of acetyl-CoA is carbohydrates, which are broken down by glycolysis to produce pyruvate. Pyruvate, in turn, is de^{c}arboxylated by the enzyme pyruvate dehydrogenase. The decarbox_{y}lated pyruvate generates acetyl-CoA, according to the following equation:

CH₃C(═O)C(═O)O⁻+HSCoA+NAD⁺→CH₃C(═O)SCoA+NADH+H⁺+CO₂

where CH₃C(═O)C(═O)O⁻ represents pyruvate; and
CH₃C(═O)SCoA represents acetyl-CoA.

Regulation of the Krebs cycle is largely dependent upon product inhibition and substrate availability. For example, NADH, a product of all dehydrogenases in the cycle (with the exception of succinate dehydrogenase) inhibits pyruvate dehydrogenase, isocitrate dehydrogenase, α-ketoglutarate dehydrogenase, and citrate synthase. Acetyl-coA inhibits pyruvate dehydrogenase, while succinyl-CoA inhibits alpha-ketoglutarate dehydrogenase and citrate synthase.

Various elements and compositions have thus been employed in an effort to modulate one or more Krebs cycle processes and, thereby, enhance the generation of ATP. For example, calcium has been successfully employed to regulate the Krebs cycle. Calcium activates pyruvate dehydrogenase phosphatase, which, in turn, activates the pyruvate dehydrogenase complex. Calcium also activates isocitrate dehydrogenase and α-ketoglutarate dehydrogenase. This increases the reaction rate of many of the sequences in the cycle, and therefore increases flux throughout the pathway.

Citrate has also been employed as a feedback inhibitor. Citrate inhibits phosphofructokinase, i.e. an enzyme involved in glycolysis that catalyses formation of fructose 1,6-bisphosphate, which is a precursor of pyruvate. This inhibits the formation of a high rate of flux when there is an accumulation of citrate.

Recent efforts have also been directed to the link between intermediates of the Krebs cycle and the regulation of hypoxia-inducible factors (HIF). HIF plays a role in the regulation of oxygen homeostasis, and is a transcription factor that targets angiogenesis, vascular remodeling, glucose utilization, iron transport and apoptosis.

HIF is synthesized consititutively. Hydroxylation of at least one of two critical proline residues also mediates their interaction with the von Hippel Lindau E3 ubiquitin ligase complex, which targets them for rapid degradation. This reaction is catalyzed by prolyl 4-hydroxylases.

Various elements and compositions, such as fumarate and succinate, have thus been employed in an effort to inhibit the formation of prolyl hydroxylases and, thereby stabilize HIF.

Although some of the noted elements and compositions have garnered some success in modulating the Krebs cycle and, thereby enhancing the available ATP energy, there is a need to provide improved agents and compositions, i.e. biochemical scaffolds, which modulate multiple Krebs cycle reactions and/or pathways.

It would thus be desirable to provide improved agents and compositions, i.e. biochemical scaffolds, which modulate multiple Krebs cycle reactions and/or pathways.

It would also be desirable to provide biochemical scaffolds that modulate additional cellular activities and/or the proliferation of bioactive agents to enhance cell function and, thereby, biological functioning, e.g., endurance, mental acuity, etc.

It is therefore an object of the present invention to provide biochemical scaffolds that modulate at least one Krebs cycle metabolic reaction, process and/or pathway.

It is another object of the present invention to provide biochemical scaffolds that modulate multiple Krebs cycle metabolic reactions, processes and/or pathways.

It is another object of the present invention to provide biochemical scaffolds that modulate oxidative stress and, thereby, mitochondria function.

It is another object of the present invention to provide biochemical scaffolds that enhance ATP energy.

It is another object of the present invention to provide biochemical scaffolds that induce the generation or proliferation of glutathione and/or a member of the glutathione family.

It is another object of the present invention to provide biochemical scaffolds that induce the generation or proliferation or communication of at least one neurotransmitter.

It is another object of the present invention to provide biochemical scaffolds that protect and/or facilitate the repair of mitochondrial DNA.

It is another object of the present invention to provide biochemical scaffolds that enhance physical endurance and mental acuity.

### SUMMARY OF THE INVENTION

The present invention is directed to biochemical scaffolds and associated methods for modulating at least one, more preferably, a plurality of cell activities and functions, including, without limitation, (i) at least one Krebs cycle metabolic reaction, process and/or pathway, (ii) generation or proliferation of glutathione and/or a member of the glutathione family, (ii) generation or proliferation or communication of at least one neurotransmitter, or inhibits the generation or proliferation or communication of selective neurotransmitters and/or processes associated therewith, and/or (iv) facilitates the repair of mitochondrial DNA.

In some embodiments of the invention, the biochemical scaffolds comprise two platforms: a vibrational energy platform and a bioenergetic platform.

In a preferred embodiment of the invention, the vibrational energy platform comprises laser activated biologically targeted energy blanks or signatures.

In a preferred embodiment, the bioenergetic platform comprises a specially formulated complex proprietary liquid herbal blend, i.e. a tincture, of oxygen enriched glycerin infused water molecules, and a specific assortment of complex-B vitamins, which helps aid the flow and the transformations of energy that occur in living organisms.

In a preferred embodiment of the invention, the bioenergetic platform comprises a Krebs cycle modulator, glutathione modulator, neurotransmitter modulator and DNA modulator.

In some embodiments of the invention, the Krebs cycle modulator is selected from the group comprising, without limitation, eleuthero root (or extract), maca, an amino acid, e.g., L-arginine and L-citrulline, and vitamins B₂, B₁, B₃, B₅ and B₉.

In some embodiments of the invention, the glutathione modulator induces the generation or proliferation of glutathione and/or a member of the glutathione family, including, without limitation, glutathione peroxidase.

In some embodiments, the gluₜathᵢone mo_{du}lator induces catalase synthesis.

In some embodiments of the invention, the glutathione modulator is selected from the group comprising, without limitation, schisandra chinensis berry, damiana and epimedium, maca, nettle leaf, Fe and Cu, and B-vitamins selected from the group comprising B₂, B₅, B₆ and B₇.

In some embodiments of the invention, the neurotransmitter modulator is selected from the group comprising, without limitation, nettle leaf, maca, eleuthero root, Yohimbe, and vitamins B₁ and B₆.

In some embodiments of the invention, the DNA modulator comprises, without limitation, vitamin B₁₂.

In some embodiments, the bᵢoenₑrgeₜic plaₜform includes a cofactor, including, without limitation, organic cofactors, such as _{f}lavin and heme, and inorganic cofactor, su_{c}h as tₕe metal ions Mg²⁺, Cu⁺, Mn²⁺, and iron-sulfur clusters.

In a preferred embodiment of the invention, the vibrational energy platform comprises energy signature extracts from a combination of herbs selected from the group comprising, without limitation, Schisandra Chinensis, Damiana Leaf, Eleuthero Root, Stinging Nettle Leaf, Maca Root, Yohimbe Root, Epimedium, L-Arginine, and L-Citrulline, which, according to the invention, help to counteract free-radical effects on cellular respiration.

In some embodiments, the biochemical platforms further include a pharmacological platform.

In some embodiments, the pharmacological platform includes one or more additional bioactive agents, such as a pharmacological agent or drug.

According to the invention, the biochemical platforms can be delivered to host tissue by various conventional means, including, without limitation, oral, sublingual, nasal, direct injection, topical application, etc.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a Krebs cycle; and

FIG. 2 is a schematic illustration of creatine phosphate—ATP interaction.

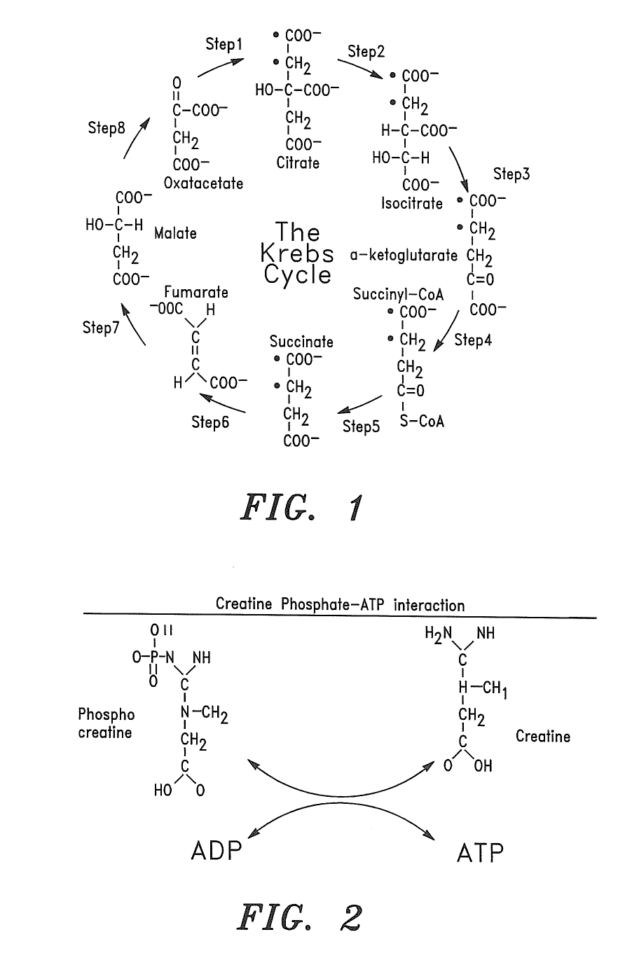

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

### Definitions

The term “vibrational energy platform,” as used herein, means and includes biologically targeted complex, stable, and efficient energetic blanks and glycerol water-soluble molecules, which, when programmed with a laser charged imprint of herbs, minerals, vitamins, amino acids, or pharmaceutical properties (creating energy-signature templates), help stimulate/enable/enhance vital cellular biochemical processes necessary to maintain homeostasis.

The term “Krebs cycle modulator,” as used herein, means and includes an element, agent, drug, compound, composition of matter or mixture thereof, including its formulation, which modulates a Krebs cycle metabolic reaction, process and/or pathway, including, without limitation, Krebs cycle product inhibition and/or substrate availability. Suitable Krebs cycle modulators include, without limitation, eleuthero root (or extract), maca, an amino acid, e.g., L-arginine and L-citrulline, and vitamins B₂, B₁, B₃, B₅ and B₉ vitamins B2, B1, B3, B5 and B9.

The term “neurotransmitter modulator,” as used herein, means and includes an element, agent, drug, compound, composition of matter or mixture thereof, including its formulation, which induces the generation or proliferation or communication of at least one neurotransmitter, or inhibits the generation or proliferation or communication thereof. Suitable neurotransmitter modulators include, without limitation, nettle leaf, maca, eleuthero root, Yohimbe, and vitamins B₁ and B₆.

The term “glutathione mo_{d}ulaₜor,” as used hₑᵣein, means and includes an element, agent, drug, compound, composition of matter or mixture thereof, including its formulation, which induces the generation or proliferation of glutathione and/or the glutathione family, including, without limitation, glutathione peroxidase.

The term “glutathione mₒdulₐtor” alₛo meanₛ and includes an element, agent, drug, compound, composition of matter or mixture thereof, including its formulatioₙ, whicₕ induces catalase synthesis.

Suitable glutathione modulators include, without limitation, schisandra chinensis berry, damiana and epimedium, maca, nettles leaves, Fe and Cu, and B-vitamins selected from the group comprising B₂, B₅, B₆ and B₇.

The term “DNA modulator,” as used herein, means and includes an element, agent, drug, compound, composition of matter or mixture thereof, including its formulation, which protects or facilitates the repair of mitₙchoₙdrial _{D}NA. A suitable DNA modulator includes, without limitation, vitamin B₁₂.

The terms “cellular dysfunction” and “cell dysfunction” are used interchangeably herein and mean and include a reduction or impairment in physical structure or function of a cell.

The term “organ dysfunction”, as used herein, means and includes a reduction or impairment in physical structure or function of a mammalian organ, including, without limitation, the cardiovascular vascular system (heart and lungs), digestive system (salivary glands, esophagus, stomach, liver, gallbladder, pancreas, intestines, colon, rectum and anus), endocrine system (hypothalamus, pituitary gland, pineal body, thyroid, parathyroids and adrenals), excretory system (kidneys, ureters, bladder and urethra), immune system (lymphatic system, tonsils, adenoids, thymus and spleen), integumentary system (skin, hair and nails), muscular system, nervous system (brain and spinal cord), reproductive system (ovaries, fallopian tubes, uterus, vagina, mammary glands, prostate and penis), respiratory system (pharynx, larynx, trachea, bronchi and diaphragm) and the skeletal system (bones, cartilage, ligaments and tendons).

The terms “prevent” and “preventing” are used interchangeably herein, and mean and include reducing the frequency or severity of a disease, condition, dysfunction or disorder. The term does not require an absolute preclusion of the disease, condition, dysfunction or disorder. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition, dysfunction or disorder. The terms include “active treatment”, i.e. treatment directed specifically toward the improvement of a disease, pathological condition, dysfunction or disorder, and “causal treatment”, i.e. treatment directed toward removal of the cause of the associated disease, pathological condition, dysfunction or disorder.

The terms “treat” and “treatment” further include “palliative treatment”, i.e. treatment designed for the relief of symptoms rather than the curing of the disease, pathological condition, dysfunction or disorder, “preventative treatment”, i.e. treatment directed to minimizing or partially or completely inhibiting the development of the associated disease, pathological condition, dysfunction or disorder, and “supportive treatment”, i.e. treatment employed to supplement another specific therapy directed toward the improvement of the associated disease, pathological condition, dysfunction or disorder.

The terms “pharmacological agent,” “active agent” and “drug” are used interchangeably herein, and mean and include an agent, drug, compound, composition Of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent,” “active agent” and “drug” thus mean and include, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, and vasodilating agents.

The term “therapeutically effective”, as used herein, means that the amount of a Krebs cycle modulator, glutathione modulator, neurotransmitter modulator or DNA modulator and/or biochemical scaffold formed therefrom or pharmacological or bioactive agent administered to a subject is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “delivery” and “administration” are used interchangeably herein, and mean and include providing a Krebs cycle modulator, glutathione modulator, neurotransmitter modulator or DNA modulator and/or biochemical scaffold formed therefrom to a subject through any method appropriate to deliver formulations and/or scaffolds to a subject. Non-limiting examples of delivery methods include oral, sublingual, nasal, direct injection, topical application, etc.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As indicated above, the present invention is directed to biochemical scaffolds and associated methods for modulating at least one, more preferably, a plurality of cell activities and functions, including, without limitation, (i) at least one Krebs cycle metabolic reaction, process and/or pathway, (ii) generation or proliferation of glutathione and/or a member of the glutathione family, (ii) generation or proliferation or communication of at least one neurotransmitter, or inhibits the generation or proliferation or communication of selective neurotransmitters and/or processes associated therewith, and/or (iv) repair of mitochondrial DNA. The noted modulated cell activities and functions result in enhanced cellular function and, thereby, musculature performance, endurance and/or stamina, and mental acuity.

In a preferred embodiment of the invention, the biochemical scaffolds comprise two platforms: a vibrational energy platform and a bioenergetic platform.

In a preferred embodiment of the invention, the vibrational energy platform comprises laser activated biologically targeted energy blanks.

In a preferred embodiment, the bioenergetic platform comprises a specially formulated complex proprietary liquid herbal blend, i.e. a tincture, of oxygen enriched glycerin infused water molecules, and a specific assortment of complex-B vitamins, which helps aid the flow and the transformations of energy that occur in living organisms.

In a preferred embodiment of the invention, the bioenergetic platform comprises a Krebs cycle modulator, glutathione modulator, neurotransmitter modulator and DNA modulator.

In some embodiments of the invention, the Krebs cycle modulator is selected from the group comprising, without limitation, eleuthero root (or extract), maca, an amino acid, e.g., L-arginine and L-citrulline, and vitamins B₂, B₁, B₃, B₅ and B₉.

In some embodiments of the invention, the glutathione modulator induces the generation or proliferation of glutathione and/or a member of the glutathione family, including, without limitation, glutathione peroxidase.

In some embodiments, the glutathione modulator induces catalase synthesis.

In some embodiments of the invention, the glutathione modulator is selected from the group comprising, without limitation, schisandra chinensis berry, damiana and epimedium, maca, nettle leaf, Fe and Cu, and B-vitamins selected from the group comprising B₂, B₅, B₆ and B₇.

In some embodiments of the invention, the neurotransmitter modulator is selected from the group comprising, without limitation, nettle leaf, maca, eleuthero root, Yohimbe, and vitamins B₁ and B₆.

In some embodiments of the invention, the DNA modulator comprises, without limitation, vitamin B₁₂.

In some embodiments, the bioenergetic platform includes a cofactor, including, without limitation, organic cofactors, such as flavin and heme, and inorganic cofactor, such as the metal ions Mg²⁺, Cu⁺, Mn²⁺, and iron-sulfur clusters.

In a preferred embodiment of the invention, the vibrational energy platform comprises a combination of herbs selected from the group comprising, without limitation, Schisandra Chinensis, Damiana Leaf, Eleuthero Root, Stinging Nettle Leaf, Maca Root, Yohimbe Root, Epimedium, L-Arginine, and L-Citrulline, which, according to the invention; help to counteract free-radical effects on cellular respiration.

In some embodiments, the biochemical platforms include one or more additional bioactive agents.

As also indicated above and discussed in detail herein, in a preferred embodiment of the invention, the biochemical scaffolds modulate multiple Krebs cycle reactions and/or pathways, resulting in enhanced adenosine-5′-triphosphate (ATP) energy potential. The biochemical scaffolds also modulate the rate/efficiency of cellular respiration provided by mitochondria.

As is well known in the art, ATP is a multifunctional nucleoside triphosphate that is used as a coenzyme in cells. ATP is one of the end products of photophosphorylation and cellular respiration, and is used by structural proteins in many cellular processes, including biosynthetic reactions, motility, and cell division.

Mammalian mitochondria are organelles that produce more than 90% of cellular ATP. In addition to supplying ATP, i.e. cellular energy, mitochondria are also involved in other cellular mechanisms, including cellular differentiation, apoptosis, as well as cell cycle modulation and cell growth.

Mitochondria provide intracellular ATP via a process called glycolysis, which breaks down monosaccharides into ATP through a series of biochemical processes. Mitochondria contain, among other things, the Krebs cycle enzymes that are involved in heme biosynthesis and the electron transport chain, i.e. the Oxidative Phosphorylation pathway (OxPHOS) system. Due to the large flux of redox reactions necessary to maintain oxidative phosphorylation, mitochondria are the primary site of production of reactive oxygen species (ROS).

It has, however, been found that increased production of ROS and interference with the OxPhos system can cause cell cycle dysfunction and arrest.

The OxPHOS system is composed of five large multi-protein enzyme complexes, which collectively transform the reducing energy of NADH and FADH₂ to ATP. NADH ubiquinone oxidoreductase (Complex I) contains 45 different subunits, and succinate ubiquinone reductase (Complex II), ubiquinone-cytochrome c oxidoreductase (Complex III), cytochrome c oxidase (Complex IV) and the ATP synthase (Complex V) have 4, 11, 13 and 16 subunits, respectively.

Four of the OxPHOS enzyme complexes (Complexes I, III, IV and V) have a dual genetic origin, i.e. they are composed of both nuclear DNA-encoded proteins and mitochondrial DNA-encoded proteins.

Transient ischemia (anoxia) results in the local production of extremely high levels of ROS, which can cause long term damage to mitochondria. In the initial phase of transient ischemia, oxygen is scarce, but tissue demands for ATP remain high, resulting in continued functioning of the OxPhos system except for the terminal reduction of oxygen to water by Complex IV. Therefore, reduced electron acceptors “upstream” of Complex IV accumulate to abnormally high levels.

Upon resupply of oxygen, these excess reduced carriers react directly with oxygen to generate highly toxic partially reduced oxygen species, which are capable of protein, lipid and DNA modifying reactions. The resulting oxidative damage is deemed to occur mainly inside the mitochondrion, because such radicals are so reactive that they are short lived and cannot diffuse far before finding a target for reaction.

Accordingly, OxPHOS proteins and intDNA are deemed the cellular molecules most affected by such oxidative stress. The resulting defects in intDNA and OxPHOS proteins can, and in most instances will, result in continued increased production of ROS.

However, by modulating the OxPhos system and, thereby, ROS production, which can be achieved by the biochemical scaffolds of the invention, oxidative stress of cells can be substantially reduced or eliminated.

As discussed in detail below, in some embodiments of the invention, the biochemical scaffolds are selected based on anticipated interaction(s) with the cellular respiration process and free radical, i.e. ROS, binding.

The selection, function and synergistic relationship by and between the scaffold platforms and modulators of the invention will now be described in detail.

### Vibrational Energy Platform

According to the invention, cellular transformation is measured by mathematical computations of the increased percentages (if any) of both pre- and post-oxygen and ATP levels with respect to each layered vibrational energy platform (VEP) application.

By way of example, for a diatomic molecule, the vibrational element can be approximated by the quantum harmonic oscillator, where the vibrational energy Ev is determined as follows:

Ev=(v+1/2)hv0,

where
v is an integer representing vibrational quantum numbers such that v=0,1,2,3, . . . , where v=0 for a diatomic molecule at the ground vibrational state;
h is Planck's constant; and
v0 is the natural frequency of the harmonic oscillator.

Further, a diatomic molecule can be represented by the difference between the energy of the molecule idealized by setting the rotational energy equal to zero, and that of a further idealized molecule which is obtained by gradually stopping the vibration of the nuclei without placing any new constraint on the motions of electrons.

Another way a diatomic molecule can move is to have each atom oscillate or vibrate along a line (the bond) connecting the two atoms. The vibrational energy is approximately that of a quantum harmonic oscillator.

According to the invention, the herbs of the invention are chosen for their synergistic and intrinsic values to be of catalytic benefit in the cellular respiration process to produce adequate oxygen in the cell, thereby being of catalytic benefit in the production of the necessary energy (ATP).

As indicated above, the herbs can comprise, without limitation, Schisandra Chinensis, Damiana Leaf, Eleuthero Root, Stinging Nettle Leaf, Maca Root, and Yohimbe Root.

The biologically targeted energetic blanks of the invention are sufficiently stable, whereby they do not require follow-up or periodic percussion, as the energetic blank's intrinsic qualities maintains the integrity of the programmed energy signature(s) intact. Energetic blanks are also so stable that an energy signature programmed into the blank can go through an airport x-ray machine, or pass by an operating micro-wave oven, or be in close proximity to an electro-magnetic field and the programmed energy signature remains intact and coherent.

According to the invention, the energetic blanks can be programmed into bio-energetic blanks in a manner that is similar to that of any other medium (scaffold/blank) used to hold an energy signature with one notable difference: Programmers need not be concerned with the environmental or ambient conditions in the same manner as is done in preparing typical energetic preparations. This is due, in large part, to the highly structured and super stable nature of energetic blanks.

In one embodiment of the invention, the energetic blanks are generated as follows:

A composition, i.e. biochemical scaffold, comprising herbal menstruums (HM) and liquid vitamin B-complex menstruums is initially prepared. In this embodiment, the HMs comprise schisandra chinensis, epimedium, damiana leaf, yohimbe, maca root, nettle leaf, eleuthero root, L-arginine and L-citrulline, and the VMs comprise vitamin B-1, B-2, B-3, B-5, B-6, B-7, B-9 and B-12.

The wt. of the VMs per dose, which, according to the invention, is typically in the range of 0.006-0.070 oz, is as follows: niacin-4 mg/dose; niacinimide-4 mg/dose; riboflavin-2.5 mg/dose; thiamin-5 mg/dose; pantothenic acid-2.5 mg/dose; pyridoxine-2.5 mg/dose; biotin-500 mcg/dose; folic acid-500 mcg/dose; and methylcobolamine-800 mcg/dose.

After the noted scaffold is prepared, the scaffold is subjected to the following steps:
1. Combine HM and ESB in a first container;
2. Oscillate HM/ESB mixture for 3-5 hours at a frequency ranging from approximately 102 GHz-250 GHz to obtain a positive charge;
3. Verify (record) the imprinted charge;
4. If no positive charge is recorded, steps 2 and 3 are repeated (adding incremental time as needed, e.g., from 3-4 hours, or 4-5 hours) until a positive charge is detected.
5. Combine VM and ESB in a second container;
6. Oscillate VM/ESB mixture for 30 minutes-3 hours at a frequency ranging from approximately 102 GHz-250 GHz to obtain a positive charge;
7. Verify (record) the imprinted charge;
8. If no positive charge is recorded, steps 6 and 7 are repeated until a positive charge is detected (adding incremental time as needed, e.g., from 30 minutes to 1-hour, 1-2 hours, 2-3 hours);
9. Fill a third container with the positive charged liquid of HM and VM (preferably a ratio of about 2:1);
10. Flavor with desired flavorings, e.g., drops of berry and honey flavonoids;
11. Cap and place third container in a quantum-harmonic oscillator;
12. Oscillate the combined liquids for approximately 3 hours at a frequency ranging from approximately 102 GHz-250 GHz; and
13. Store the positive charged composition in cool dry location for up to 4 weeks to allow the composition to set.

Referring now to Tables I and II, there is shown preferred harmonic oscillation-variances for herbs and B-vitamins that can be employed in the biochemical scaffolds of the invention.

TABLE I

| Herb/Component | Frequency | Time |
|---|---|---|
| Schisandra Chinensis Berry | 102 GHz-250 GHz | 3-5 Hrs |
| Damiana Leaf | 102 GHz-250 GHz | 3-5 Hrs |
| Eleuthero Root | 102 GHz-250 GHz | 3-5 Hrs |
| Stinging Nettle Leaf | 102 GHz-250 GHz | 3-5 Hrs |
| Maca Root | 102 GHz-250 GHz | 3-5 Hrs |
| Yohimbe Root | 102 GHz-250 GHz | 3-5 Hrs |
| Epimedium | 102 GHz-250 GHz | 3-5 Hrs |
| L-Arginine | 102 GHz-250 GHz | 3-5 Hrs |
| L-Citrulline | 102 GHz-250 GHz | 3-5 Hrs |

TABLE II

| Vitamin | Frequency | Time |
|---|---|---|
| B1: Thiamin | 105 GHz-250 GHz | 10 Min.-3 Hrs |
| B2: Riboflavin | 105 GHz-250 GHz | 10 Min.-3 Hrs |
| B3: Niacin/Niacinimide | 105 GHz-250 GHz | 10 Min.-3 Hrs |
| B5: Pantothenic Acid | 105 GHz-250 GHz | 10 Min.-3 Hrs |
| B6: Pyridoxine | 105 GHz-250 GHz | 10 Min.-3 Hrs |
| B7: Biotin | 105 GHz-250 GHz | 10 Min.-3 Hrs |
| B9: Folic Acid | 105 GHz-250 GHz | 10 Min.-3 Hrs |
| B12: Methylcobalamine | 105 GHz-250 GHz | 10 Min.-3 Hrs |

### Bioenergetic Platform

As indicated above, the bioenergetic platform comprises at least one Krebs cycle modulator, glutathione modulator, neurotransmitter modulator or DNA modulator.

In a preferred embodiment of the invention, the bioenergetic platform comprises a Krebs cycle modulator, glutathione modulator, neurotransmitter modulator and DNA modulator.

Each of the noted modulators are discussed in detail below.

### Krebs Cycle Modulators

As indicated above, a seminal process associated with the Krebs cycle is the catabolism of carbohydrates, fats and proteins, which results in the production of a two carbon organic product, i.e. acetate in the form of acetyl-CoA. Acetyl-CoA and two equivalents of water (H₂O) are consumed during the Krebs cycle, producing two equivalents of carbon dioxide (CO₂) and one equivalent of HS-CoA.

In addition, one complete cycle of the Kreb cycle converts three equivalents of nicotinamide adenine dinucleotide (NAD⁺) into three equivalents of reduced NAD⁺ (NADH), one equivalent of ubiquinone (Q) into one equivalent of reduced ubiquinone (QH₂), and one equivalent each of guanosine diphosphate (GDP) and inorganic phosphate (Pᵢ) into one equivalent of guanosine triphosphate (GTP). The NADH and QH₂ generated during the Kreb cycle are in turn used by the oxidative phosphorylation pathway to generate energy-rich adenosine triphosphate (ATP).

A primary source of acetyl-CoA is carbohydrates, which are broken down by glycolysis to produce pyruvate. Pyruvate is decarboxylated by the enzyme pyruvate dehydrogenase to generate acetyl-CoA.

Regulation of the Krebs cycle is largely dependent upon product inhibition and substrate availability. For example, NADH, a product of all dehydrogenases in the cycle (with the exception of succinate dehydrogenase) inhibits pyruvate dehydrogenase, isocitrate dehydrogenase, α-ketoglutarate dehydrogenase, and citrate synthase. Acetyl-coA inhibits pyruvate dehydrogenase, while succinyl-CoA inhibits alpha-ketoglutarate dehydrogenase and citrate synthase.

As indicated above, the Krebs cycle modulators of the invention are capable of modulating at least one Krebs cycle metabolic reaction, process and/or pathway, including, without limitation, product inhibition and/or substrate availability.

According to the invention, the Krebs cycle modulators can comprise, without limitation, eleuthero root, maca, an amino acid, e.g., L-arginine and L-citrulline, and vitamins B₂, B₁, B₃, B₅ and B₉.

In some embodiments, the Krebs cycle modulators of the invention modulate product and/or substrate availability. By way of example, in some embodiments, the Krebs cycle modulator comprises eleuthero root, which Applicant has found facilitates the formation of glucose 6 phosphate. As stated, glucose 6 phosphate eventually converts to pyruvate, which enters into the Krebs cycle as Acetyl-coA.

Eleuthero root also enhances the activity of succinate dehydrogenase, an enzyme that facilitates the formation of FAD to FADH₂. These processes aid in the generation of ATP.

In some embodiments, the Krebs cycle modulator comprises maca. According to the invention, maca works synergistically with eleuthero root by inducing co-factor proliferation, which supports activation of the Krebs cycle.

Maca also facilitates the production of super oxide dismutase, i.e. an important antioxidant. Intracellular super oxide dismutase converts a highly undesirable free radical known as superoxide to hydrogen peroxide and oxygen.

In some embodiments of the invention, the Krebs cycle modulator comprises an amino acid selected from the group comprising, without limitation, L-arginine and L-citrulline. Applicant has found that L-arginine and L-citrulline facilitate the production of nitrous oxide. Nitrous oxide induces vasodilation and, hence, enhanced blood flow. The enhanced blood flow results in an increase in delivered O₂ and, thereby, enhanced cellular energy.

In some embodiments of the invention, the Krebs cycle modulator comprises a B-vitamin selected from the group comprising, without limitation B₁, B₂, B₃, B₅ and B₉.

B₁, i.e. thiamine, plays a central role in the generation of energy from carbohydrates. It is involved in RNA and DNA production, as well as nerve function. B1's active form is a coenzyme called thiamine pyrophosphate (TPP), which converts pyruvate to acetyl Coenzyme A (CoA).

B₂, i.e. riboflavin, is involved in energy production for the electron transport chain and catabolism of fatty acids, i.e. beta oxidation.

B₃, i.e. niacin, is composed of two co-enzyme forms of niacin: nicotinamide adenine dinucleotide (NAD) and nicotinamide adenine dinucleotide phosphate (NADP). Both play an important role in energy transfer reactions in the metabolism of glucose, fat and alcohol.

NAD carries H₂ and associated electrons during metabolic reactions, including the pathway from the Krebs cycle to the electron transport chain. NADP is a key coenzyme in lipid and nucleic acid synthesis.

B₅, i.e. pantothenic acid, is also involved in the oxidation of fatty acids and carbohydrates. Coenzyme A, which can be synthesised from panothenic acid, is involved in the synthesis of amino acids, fatty acids, ketones, cholesterol, phospholipids, steroid hormones, neurotransmitters, such as acetylcholine, and antibodies.

B₉, i.e. folic acid, acts as a co-enzyme in the form of tetrahydrofolate (THF), which is involved in the transfer of single-carbon units in the metabolism of nucleic acids and amino acids. THF is involved in pyrimidine nucleotide synthesis, which is required for normal cell division. Folate also aids in erythropoiesis, i.e. the production of red blood cells.

### Glutathione Modulator

Glutathione; specifically, glutathione peroxidase, is an important intracellular antioxidant that induces conversion of hydrogen peroxide to H₂O and O₂. Glutathione reduces disulfide bonds formed within cytoplasmic proteins to cysteines by serving as an electron donor. In the process, glutathione is converted to its oxidized form glutathione disulfide (GSSG), as known as L-(−)-glutathione.

After oxidation, glutathione is reduced back to glutathione reductase, using NADPH as an electron donor.

As indicated above, the glutathione modulators of the invention induce the generation or proliferation of glutathione and/or a member of the glutathione family, including, without limitation, glutathione peroxidase.

According to the invention, the glutathione modulators can comprise, without limitation, schisandra chinensis berry, damiana and epimedium, and vitamin B₂. Applicant has found that each of the noted modulators enhance the production of glutathione and/or a member of the glutathione family.

As indicated above, B₂, i.e. riboflavin, facilitates energy production for the electron transport chain and catabolism of fatty acids, i.e. beta oxidation.

As also indicated above, in some embodiments of the invention, the glutathione modulator is effective to induce the synthesis of catalase, another key antioxidant. In these embodiments, the glutathione modulator can comprise, without limitation, maca, nettles leaves, Fe and Cu, and B-vitamins selected from the group comprising B₂, B₅, B₆ and B₇.

B₆, i.e. pyridoxine, is stored in the body as pyridoxal 5′-phosphate (PLP), which is the co-enzyme form of vitamin B₆. Pyridoxine is also involved in the metabolism of amino acids and lipids; in the synthesis of neurotransmitters and hemoglobin, as well as in the production of nicotinic acid (vitamin B₃). Pyridoxine also plays an important role in gluconeogenesis.

B7, i.e. biotin, also plays a key role in the metabolism of lipids, proteins and carbohydrates. It is a critical co-enzyme of four carboxylases: acetyl CoA carboxylase, which is involved in the synthesis of fatty acids from acetate; propionyl CoA carboxylase, which is involved in gluconeogenesis; β-methylcrotonyl Coa carboxylase, which is involved in the metabolism of leucin; and pyruvate CoA carboxylase, which is involved in the metabolism of energy, amino acids and cholesterol.

### Neurotransmitter Modulators

Neurotransmitters are endogenous chemicals that transmit signals from a neuron to a target cell across a synapse. The major neurotransmitters comprise various amino acids, including glutamate, aspartate, D-serine, γ-aminobutyric acid (GABA) and glycine, monomines and other biogenic amines, including dopamine (DA), norepinephrine (noradrenaline; NE, NA), epinephrine (adrenaline), histamine and serotonin (SE, 5-HT), peptides, including somatostatin, substance P, opioid peptides, as well as acetylcholine (ACh), adenosine, anandamide and nitric oxide.

Acetylcholine is an important neurotransmitter. Acetylcholine stimulates the central nervous system to enhance mental acuity, i.e. learning ability, short term memory and mental focus.

Acetylcholine is distinguished as the transmitter at the neuromuscular junction connecting motor nerves to muscles. The paralytic arrow-poison curare acts by blocking transmission at these synapses. Acetylcholine also operates in many regions of the brain, but using different types of receptors, including nicotinic and muscarinic receptors.

Dopamine is another important neurotransmitter. Dopamine works both as an inhibitory and excitatory neurotransmitter. As inhibitory neurotransmitter, it causes balance and general sense of well-being. As excitatory neurotransmitter, it improves cognition, concentration and focus.

Norepinephrine is another key neurotransmitter, which effects cognition, mood and mental concentration.

As indicated above, the neurotransmitter modulators of the invention induce the generation or proliferation or communication of at least one neurotransmitter, e.g. dopamine, norepinephrine, etc. or inhibit the generation or proliferation (or communication) of selective neurotransmitters and/or processes associated therewith.

According to the invention, the neurotransmitter modulators of the invention can comprise, without limitation, nettle leaf, maca, eleuthero root, Yohimbe, and vitamins B₁ and B₆.

Applicant has found that nettle leaf increases the amount of neurotransmitters available to act on the neuron receptors; particularly, dopamine and acetylcholine, thus improving several mental processes, e.g. learning and recollection abilities.

Maca supports acetyl cholinesterase and, thereby, similarly enhances the proliferation of acetylcholine.

In addition to the Krebs cycle functions discussed above, eleuthero root enhances neuron activities, e.g. short term memory.

Yohimbe is a pre- and post-synaptic, alpha 2 adrenergic blocker that enhances neurotransmitter release and, thereby, enhanced cognitive functioning.

Yohimbe also induces elevation of norepinephrine from the locus coeruleus, resulting in enhanced memory. It is also likely that Yohimbe can reverse recall issues associated with post traumatic stress disorder.

The synergistic effect by and between maca and eleuthero root also provides cellular balance and decreases the negative effects of stress.

### DNA Neurotransmitter Modulators

As indicated, mammalian mitochondria are organelles that produce more than 90% of cellular ATP. In addition to supplying ATP, i.e. cellular energy, mitochondria are also involved in other cellular mechanisms, including cellular differentiation, apoptosis, as well as cell cycle modulation and cell growth.

As also indicated above, when a cell has temporarily or reversibly stopped dividing or regenerating it is often deemed to have entered a quiescent or senescent state referred to as the G₀ phase.

Non-proliferative cells generally enter the senescent G₀ phase or state from the G₁ phase and may remain senescent for long periods of time, possibly indefinitely (as is often the case for neurons). This is very common for cells that are fully differentiated.

Cellular senescence was first described by Hayflick and Moorhead (1961) when they observed that normal human fibroblasts entered a state of irreversible growth arrest after serial passage in vitro. In contrast, abnormal cells, such as cancer cells, did not enter this growth arrested state and proliferated indefinitely.

The maximum number of cell divisions that a cell can undergo, termed the Hayflick limit, varies from cell type to cell type and organism. In fibroblasts, this number is about 50 divisions, after which cell division ceases.

However, some cells become senescent after fewer replication cycles as a result of DNA damage or degradation, e.g., DNA mutations, DNA oxidation and chromosome losses, which would make a cell's progeny nonviable. If the DNA damage cannot be easily repaired, the cells either prematurely age or self-destruct (i.e. apoptosis or programmed cell death).

The process of cellular senescence can also be triggered by several additional mechanisms, including telomere shortening (i.e. a form of DNA damage or degradation).

Telomeres consist of repetitive DNA elements at the end of linear chromosomes that protect chromosome ends from degradation and recombination. Due to DNA replication mechanisms and oxidative stress, telomeres become progressively shorter with each round of replication. As increasing numbers of cell division occur, the telomeres reach a critically short length, which present as double-stranded DNA breaks, resulting in telomere-initiated senescence.

Protecting and/or facilitating the repair of mitochondrial DNA, which can be achieved by virtue of the DNA modulators of the invention, is thus essential to achieve optimal cell function and, thereby, physiological functioning. Healthy mitochondrial DNA also provides a healthy enzymatic process, which is required for oxidative phosphorylation and, hence, continued energy production.

As indicated above, the DNA modulators of the invention protect and/or facilitate the repair of mitochondrial DNA.

According to the invention, the DNA modulator of the invention can comprise, without limitation, vitamin B₁₂.

B₁₂ supports DNA synthesis and, in some instances, inhibits megaloblastic anemia.

B₁₂ is also involved in the cellular metabolism of carbohydrates, proteins and lipids. It functions as a co-enzyme in intermediary metabolism for the methionine synthase reaction with methylcobalamin, and the methylmalonyl CoA mutase reaction with adenosylcobalamin.

The biochemical scaffolds of the invention thus modulate various seminal cell activities and functions, including (i) at least one Krebs cycle metabolic reaction, process and/or pathway, (ii) generation or proliferation of glutathione and/or a member of the glutathione family, (ii) generation or proliferation of at least one neurotransmitter, and/or (iv) the repair of mitochondrial DNA. The noted modulated cell activities and functions result in enhanced cellular function and, thereby, musculature performance, endurance and/or stamina, and mental acuity.

As is also well known in the art, muscle contraction and performance is directly dependent on O₂ and creatine phosphate.

As indicated above, L-arginine and L-citrulline, i.e. Krebs cycle modulators, facilitate the production of nitrous oxide. Nitrous oxide induces vasodilation and, hence, enhanced blood flow. The enhanced blood flow results in an increase in delivered O₂ and, thereby, enhanced cellular energy.

Glutathione; specifically, glutathione peroxidase, induces the conversion of hydrogen peroxide to H₂0 and O₂, and, hence, also increases available O₂.

Eleuthero root, another Krebs cycle modulator, also increases O₂ for cell and musculature performance.

Further, epimeduim, another glutathione modulator, facilitates the production of creatine phosphate, which regenerates ATP energy (see FIG. 2) and, thereby, privides energy to muscle cells. According to the invention, eleuthero root and epimedium synergistically augment muscle contraction and performance. The muscle contraction and performance is further enhanced by L-citrulline and L-arginine.

As is known in the art, muscle mass, strength, recovery, hypertrophy and, hence, performance is also dependent upon testosterone availability. Damiana is an aromatase inhibitor, which minimizes testosterone loss.

Maca also increase muscular hypertrophy.

The biochemical platform modulators also enhance the presence of important intracellular antioxidants that help bind free radicals, allowing the mitochondria to continue generating and providing ATP that is necessary for the high intensity exercise.

In some embodiments of the invention, the biochemical scaffold includes the vitamins and supplements set forth in Table III below:

TABLE III

| Vitamin | Milligrams | % of Oz. |
|---|---|---|
| B1 | 294-590 | 1%-2.1% of an ounce |
| B2 | 294-590 | 1%-2.1% of an ounce |
| B3 | 294-590 | 1%-2.1% of an ounce |
| B5 | 294-590 | 1%-2.1% of an ounce |
| B6 | 294-590 | 1%-2.1% of an ounce |
| B7 | 294-590 | 1%-2.1% of an ounce |
| B9 | 294-590 | 1%-2.1% of an ounce |
| B12 | 294-590 | 1%-2.1% of an ounce |
| L-Arginine | 294-2350 | 1%-8.3% of an ounce |
| L-Citrulline | 294-2350 | 1%-8.3% of an ounce |

In some embodiments of the invention, the biochemical scaffold includes the herbs set forth in Table IV below:

TABLE IV

| Herb | Milligrams | % of ounce |
|---|---|---|
| Schisandra Berry | 1175 mg-2350 mg | 4.2%-8.3% |
| Damiana Leaf | 1175 mg-2350 mg | 4.2%-8.3% |
| Eleuthero Root | 1175 mg-2350 mg | 4.2%-8.3% |
| Stinging Nettle Leaf | 1175 mg-2350 mg | 4.2%-8.3% |
| Maca Root | 1175 mg-2350 mg | 4.2%-8.3% |
| Yohimbe Root | 1175 mg-2350 mg | 4.2%-8.3% |
| Epimedium | 1175 mg-2350 mg | 4.2%-8.3% |

In some embodiments, the bioenergetic platform includes a cofactor, including, without limitation, organic cofactors, such as flavin and heme, and inorganic cofactor, such as the metal ions Mg²⁺, Cu⁺, Mn²⁺, and iron-sulfur clusters.

In some embodiments, the biochemical scaffolds further include at least 1200 mg of glycerin.

To further enhance the cellular activities and processes, in some embodiments of the invention, the biochemical platforms include an additional pharmacological platform.

In some embodiments, the pharmacological platform comprises at least one bioactive agent.

In some embodiments, the bioactive agent comprises an element, agent, drug, compound, composition of matter or mixture thereof, including its formulation, which modulates a cellular activities or process. According to the invention, such bioactive agents can comprise, without limitation, creatine and selective amino acids.

In some embodiments, the bioactive agent comprises a pharmacological agent or composition. Suitable pharmacological agents or compositions, include, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

According to the invention, the biochemical scaffolds can be delivered to host tissue by various conventional means, including, without limitation, oral, sublingual, nasal, direct injection, topical application, etc.

In a preferred embodiment, the biochemical scaffolds are in liquid form.

In a preferred embodiment, a dose of the liquid form biochemical scaffold comprises in the range of approximately range of 0.006-0.070 oz, more preferable, in the range of approximately 0.006-0.018 oz.

As will readily be appreciated by one having ordinary skill in the art, the present invention provides numerous advantages compared to prior art formulations and methods for enhancing cell function and, thereby physiological performance. Among the advantages are the following:
The provision of biochemical scaffolds that modulate multiple Krebs cycle metabolic reactions, processes and/or pathways.
The provision of biochemical scaffolds that modulate oxidative stress and, thereby, mitochondria function.
The provision of biochemical scaffolds that enhance ATP energy.
The provision of biochemical scaffolds that enhance physical endurance and mental acuity.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A biochemical scaffold for regulating mammalian cell function, comprising:
a bioenergetic platform and a vibrational platform, said bioenergetic platform comprising a Krebs cycle modulator, glutathione modulator, neurotransmitter modulator and a DNA modulator.

2. The biochemical scaffold of claim 1, wherein said Krebs cycle modulator is selected from the group consisting of eleuthero root, maca, an amino acid, and vitamins B₂, B₁, B₃, B₅ and B₉.

3. The biochemical scaffold of claim 2, wherein said amino acid is selected from the group consisting of L-arginine and L-citrulline.

4. The biochemical scaffold of claim 1, wherein said glutathione modulator is selected from the group consisting of schisandra chinensis berry, damiana, epimedium, maca, nettle leaf, Fe, Cu, and a B-vitamin, said B-vitamin being selected from the group consisting of B₂, B₅, B₆ and B₇.

5. The biochemical scaffold of claim 1, wherein said neurotransmitter modulator is selected from the group consisting of nettle leaf, maca, eleuthero root, Yohimbe, and vitamins B₁ and B₆.

6. The biochemical scaffold of claim 1, wherein said DNA modulator comprises vitamin B₁₂.

7. The biochemical scaffold of claim 1, wherein said bioenergetic platform further includes a cofactor.

8. The biochemical scaffold of claim 7, wherein said cofactor comprises an organic cofactor.

9. The biochemical scaffold of claim 8, wherein said organic cofactor is selected from the group consisting of flavin and heme.

10. The biochemical scaffold of claim 7, wherein said cofactor comprises an inorganic cofactor.

11. The biochemical scaffold of claim 10, wherein said organic cofactor comprises a metal ion selected from the group consisting of Mg²⁺, Cu⁺, Mn²⁺.

12. The biochemical scaffold of claim 1, wherein said vibrational energy platform comprises a first component selected from the group consisting of Schisandra Chinensis, Damiana Leaf, Eleuthero Root, Stinging Nettle Leaf, Maca Root, Yohimbe Root, Epimedium, L-Arginine and L-Citrullinen.

13. The biochemical scaffold of claim 12, wherein said first component is subjected to harmonic oscillation at a frequency in the range of approximately 102 GHz-250 GHz for a period of time in the range of approximately 3-5 hrs.

14. The biochemical scaffold of claim 1, wherein said vibrational energy platform comprises a second component selected from the group consisting of a B vitamin selected from the group consisting of B1, B2, B₃, B₅, B₆, B₇, B₉ and B₁₂.

15. The biochemical scaffold of claim 14, wherein said second component is subjected to harmonic oscillation at a frequency in the range of approximately 105 GHz-250 GHz for a period of time in the range of approximately 10 min.-3 hrs.
